(19) United States
(12) Patent Application Publication
(10) Pub. No.: US 2011/0060158 A1
(43) Pub. Date: Mar. 10, 2011

(54) Preparation Method for (Meth)acrylate

(75) Inventors: Hyun-Kyu Kim, Daejeon Metropolitan City (KR); Dong-Hyun Cho, Daejeon Metropolitan City (KR); Jun-Seok Ko, Daejeon Metropolitan City (KR); Kyong-Yong Cha, Daejeon Metropolitan City (KR); Se-Won Baek, Daejeon Metropolitan City (KR); Dong-Hyun Woo, Daejeon Metropolitan City (KR)

(73) Assignee: LG CHEM, LTD., Seoul (KR)

(21) Appl. No.: 12/853,430

(22) Filed: Aug. 10, 2010

(30) Foreign Application Priority Data
Sep. 8, 2009 (KR) 10-2009-0084586

(51) Int. Cl. C07C 69/54

(52) U.S. Cl. 560/205

(57) ABSTRACT

The present invention provides a method for preparing (meth)acrylate by esterification reaction of reactants containing (meth)acrylic acid and alcohol in the presence of a catalyst, the preparation method for (meth)acrylate is characterized in that a hydrazine compound or derivative thereof is put into an esterification reactor; and (meth)acrylate prepared by the same. The present invention effectively prevents impurities generated by aldehyde that is contained in the raw material (meth)acrylic acid from remaining in a production apparatus and the catalyst, so that the process for producing (meth)acrylate proceeds smoothly.

### CROSS-REFERENCE TO RELATED APPLICATION

This application claims priority from Korean Patent Application No. 10-2009-0084586 filed on Sep. 8, 2009 in the Korean Intellectual Property Office, the disclosure of which is incorporated herein by reference in its entirety.

### BACKGROUND OF THE INVENTION

The present invention relates to a preparation method for (meth)acrylate, in which the method effectively prevents impurities generated by aldehyde that is contained in the raw material (meth)acrylic acid from remaining in a production apparatus and the catalyst, so that the process for producing (meth)acrylate proceeds smoothly.

In the preparation method of (meth)acrylate, ion exchange resin, and inorganic acid, organic acid are generally used as a catalyst for esterification reaction.

In esterification reaction, the compositions of input liquid and output liquid in a reactor are analyzed to confirm the change ratio of acrylic acid. The conversion ratio of acrylic acid and the selectivity of acrylate are determined, on the basis of the peak point at which the change ratio of acrylic acid maintains constantly.

The esterification reaction using (meth)acrylic acid as a raw material is preferably carried out at 50° C. to 150° C., and more preferably 70° C. to 100° C., even though it will vary depending on the desired ester products, in which the esterification reaction is performed in a liquid phase, and the reaction may take place in either the mobile or stationary phase.

A polymerization inhibitor is typically used for esterification reaction. The useful polymerization inhibitor can be exemplified by hydroquinone, methoxy hydroquinone, phenothiazine, hydroxylamine, phenylene diamine or the like. In addition, when esterification reaction is performed in the presence of molecular oxygen, the effects of polymerization inhibitor can be improved.

As such, during the production process of (meth)acrylate, there are problems that solid or liquid impurities remain in the bottom portion or pipeline of a reactor or refiner, which reduce the process efficiency or bind to the active site of a porous catalyst to shorten its lifetime.

### SUMMARY OF TEE INVENTION

In order to solve the above problems generated in the known methods, an aspect of the present invention is to provide a preparation method for (meth)acrylate, in which the method effectively prevents impurities generated by aldehyde that is contained in the raw material (meth)acrylic acid from remaining in a production apparatus and the catalyst, so that the process for producing (meth)acrylate proceeds smoothly; and (meth)acrylate prepared by the same.

The preparation method of present invention effectively prevents impurities generated by aldehyde that is contained in the raw material (meth)acrylic acid from remaining in a production apparatus and the catalyst, so that the process for producing (meth)acrylate proceeds smoothly, and (meth)acrylate can be prepared in a high yield.

### BRIEF DESCRIPTION OF TEE DRAWINGS

FIG. 1 is a schematic diagram showing the production process of (meth)acrylate according to the present invention; and

FIG. 2-1 shows the bottom portion of a refiner after operating according to Example 6.

FIG. 2-2 shows the bottom portion of a refiner after operating according to Comparative Example 2.

In one embodiment, the present invention provides a preparation method for (meth)acrylate by esterification reaction of reactants comprising (meth)acrylic acid and alcohol in the presence of a catalyst, in which a hydrazine compound or derivative thereof is put into an esterification reactor.

In another embodiment, the present invention provides (meth)acrylate prepared by the same.

Hereinafter, the present invention will be described in more detail.

The present invention relates to a method for preparing (meth)acrylate by esterification reaction of reactants comprising (meth)acrylic acid and alcohol in the presence of a catalyst, characterized in that a hydrazine compound or derivative thereof is put into an esterification reactor.

In general, a small amount of aldehyde compound is contained in (meth)acrylic acid, and the aldehyde compound may generate impurities during the production process of (meth)acrylate.

In the present invention, the (meth)acrylic acid is intended to refer to an alkyl group having 1 to 6 carbon atoms, and encompasses substituted or unsubstituted acrylic acid or methacrylic acid. The (meth)acrylate is intended to refer to an alkyl group having 1 to 6 carbon atoms, and encompasses substituted or unsubstituted acrylate or methacrylate.

Further, the alcohol may be an alkyl group having 1 to 6 carbon atoms, and may be butanol, but is not limited thereto.

In the present invention, the impurities refer to a solid or liquid material that is formed by aldehyde compound contained in the raw material (meth)acrylic acid, and mean that they have low solubility to a reaction solvent to remain in the reactor, refiner or bottom portion of pipeline or bind to the active site of a porous catalyst to shorten its lifetime.

In order to find the materials that cause the impurities, the present inventors examined generation of the impurities while separately removing the trace compounds from the raw materials that are used for the production of (meth)acrylate.

Consequently, when the trace aldehyde compound is removed from the raw material (meth)acrylic acid, the generation of impurities was not observed, indicating that the aldehyde compound causes the generation of impurities during the production process of (meth)acrylate.

Accordingly, the generation of impurities can be prevented by using (meth)acrylic acid, from which the aldehyde compound is removed. However, the removal of aldehyde compound from (meth)acrylic acid requires an additional process. In the case of adding a hydrazine compound that is known to have reactivity with the aldehyde compound, the reaction product of aldehyde and hydrazine is formed, and thus an additional process is required to remove the reaction product, which is not preferred in economic terms.

As a method for removing the aldehyde compound, which is also preferred in economic terms, the present inventors investigated that a hydrazine compound or derivative thereof is directly added to the esterification reactor, thereby preventing impurities generated by the aldehyde compound in (meth)acrylic acid from remaining in a production apparatus and a catalyst.

Examples of the aldehyde compound that is contained in (meth)acrylic acid to cause the generation of impurities include, but are not limited to, furfural, benzaldehyde, acrolein, and mixtures thereof.

In the present invention, the hydrazine compound or derivative thereof means a compound having a hydrazine group or derivative thereof, which functions to react with the aldehyde compound to prevent the generation of impurities, thereby preventing the impurities from remaining in a production apparatus and a catalyst. Examples of the hydrazine compound or derivative thereof include, but are not limited to, hydrazine hydrate(HH), phenyl hydrazine and derivatives thereof, thiosemicarbazide, toluenesulfonohydrazide and derivatives thereof, aminoguanidine, a aminotriazole derivative, a tolylhydrzine derivative, 4-pyridine carboxylic acid hydrazide, N,N-dimethylhydrazine, benzophenone hydrazone, an aminopiperazine derivative, an aminomorpholine derivative, benzhydrazide, acetohydrazide, oxalyl dihydrazide, adipic dihydrazide, formylhydrazine, etc. and among them, hydrazine hydrate is specifically preferable.

In general, a nitrogen compound is formed by the reaction of the hydrazine compound and aldehyde compound. Therefore, the known method required a pretreatment process for separation and purification of (meth)acrylic acid from the produced nitrogen compound, when the raw material (meth)acrylic acid is treated with the hydrazine compound or derivative thereof. However, like in the present invention, when the hydrazine compound or derivative thereof is directly added to the esterification reactor containing (meth)acrylic acid or the input line of (meth)acrylic acid during the production process of (meth)acrylate, the nitrogen compound, which is produced by the reaction of (meth)acrylic acid and the hydrazine compound or derivative thereof, is dissolved in the reaction solvent or circulated in a mixed liquid state, and then discharged. Thus, the nitrogen compound does not remain in the production apparatus or the catalyst during the production process, and the final product is not deteriorated.

The hydrazine compound or derivative thereof may be directly added to the esterification reactor, or added to the pipeline, through which (meth)acrylic acid is transferred to the esterification reactor.

In the present invention, the hydrazine compound or derivative thereof is preferably added to the esterification reactor in an amount of 2 to 8 molar ratio, and more preferably 2.5 to 4 molar ratio of the aldehyde compound in (meth)acrylic acid. If the amount of the hydrazine compound or derivative thereof is within the above range, the aldehyde compound can be treated rapidly and sufficiently.

The preparation method of (meth)acrylate may be in either batch or continuous mode, and preferably in continuous mode.

In the present invention, if impurities are generated, they remain in the apparatus for producing (meth)acrylate to reduce the production efficiency, in which the production apparatus having residual impurities means the bottom portion or pipeline of the reactor and refiner.

In the present invention, the catalyst is preferably one or more selected from the group consisting of inorganic acid such as sulfuric acid, hydrochloric acid, phosphoric acid, and nitric acid; organic acid containing a sulfonic group such as methansulfonic acid, para toluenesulfonic acid(pTSA), and alkyl sulfuric acid; natural/synthetic zeolite, cation and anion exchange resins; metal salt such as lithium fluoride, potassium chloride, cesium chloride, calcium chloride, ferric chloride, and aluminum phosphates; metal oxide such as heteropoly acid; and organic metal such as tetra alkyl titanate and polymers thereof.

Among them, para toluenesulfonic acid, cation exchange resin or anion exchange resin is more preferred.

Further, the catalyst is preferably used in an amount of 0.01 to 50% by weight, based on 100% by weight of the reactants.

Hereinafter, the present invention will be described in more detail with reference to Examples. Although the Examples and Comparative Examples are limited to butyl acrylate as the desired product of esterification reaction, those skilled in the art will appreciate that the results of the following Examples and Comparative Examples can be readily applied to other types of (meth)acrylate. The following Examples are for illustrative purposes only, and the invention is not intended to be limited by these Examples.

### EXAMPLE

### Comparative Example 1

500 g of acrylic acid containing 350 ppm of furfural was added to a 1 L beaker, and stirred at room temperature. Hydrazine hydrate corresponding to 1.5 molar ratio of the total amount of furfural was added thereto. While stirring the mixture constantly, the furfural content was evaluated at each time point. The results are shown in the following Table 1.

### Example 1

The evaluation was performed in the same manner as in Comparative Example 1, except for adding hydrazine hydrate corresponding to 2 molar ratio of the total amount of furfural. The results are shown in the following Table 1.

### Example 2

The evaluation was performed in the same manner as in Comparative Example 1, except for adding hydrazine hydrate corresponding to 3 molar ratio of the total amount of furfural. The results are shown in the following Table 1.

### Example 3

The evaluation was performed in the same manner as in Comparative Example 1, except for adding hydrazine hydrate corresponding to 4 molar ratio of the total amount of furfural. The results are shown in the following Table 1.

### Example 4

The evaluation was performed in the same manner as in Comparative Example 1, except for adding hydrazine hydrate corresponding to 6 molar ratio of the total amount of furfural. The results are shown in the following Table 1.

### Example 5

The evaluation was performed in the same manner as in Comparative Example 1, except for adding hydrazine hydrate corresponding to 8 molar ratio of the total amount of furfural. The results are shown in the following Table 1.

TABLE 1

| | Furfural content in acrylic acid (ppm) | | | | | |
|---|---|---|---|---|---|---|
| | Comparative | Exam- | Exam- | Exam- | | Exam- |
| | Example 1 | ple 1 | ple 2 | ple 3 | Example 4 | ple 5 |
| 2 min | 10.7 | 2.3 | 0.2 | 0.2 | 0.1 | 0.0 |
| 30 min | 41.1 | 10.7 | 4.2 | 0.7 | 0.8 | 0.3 |
| 1 hr | 81.5 | 35.2 | 11.5 | 1.0 | 0.9 | 0.0 |
| 2 hr | 111.1 | 51.8 | 20.0 | 1.4 | 0.9 | 0.0 |
| 3 hr | — | 65.6 | 27.4 | 3.5 | 0.7 | 0.5 |
| 4 hr | — | 75.6 | 32.6 | 3.4 | 0.6 | 0.5 |

### Example 6

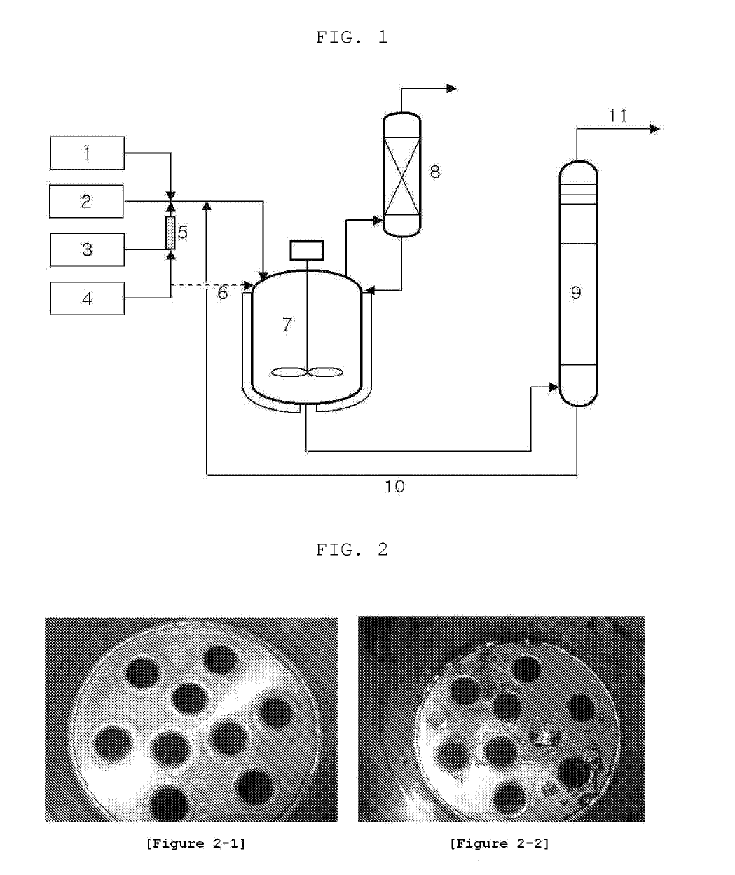

In the reaction apparatus for producing acrylate by esterification reaction of acrylic acid and butyl alcohol in the presence of a pTSA catalyst, hydrazine hydrate corresponding to 4 molar ratio of the total amount of furfural in acrylic acid was put into a pipeline between an acrylic acid tank and an ester reactor, and the acrylate reaction apparatus was operated. Then, the reactants were transferred to a purification column of a purification part, and the purification column was operated for 72 hrs. At this time, the interiors of the purification column and reboiler were examined with the naked eye. No impurities were found, as shown in FIG. 2-1.

### Comparative Example 2

The reaction apparatus was operated in the same manner as in Example 6, except for adding no hydrazine hydrate. The interiors of the purification column and reboiler were examined with the naked eye. The generation of impurities was found, as shown in FIG. 2-2.

What is claimed is:

1. A method for preparing (meth)acrylate comprising performing esterification reaction of reactants comprising (meth)acrylic acid and alcohol in the presence of a catalyst, wherein a hydrazine compound or derivative thereof is introduced into an esterification reactor during the esterification reaction step.

2. The method according to claim 1, wherein the hydrazine compound or derivative thereof is added to the esterification reactor in an amount of 2 to 8 molar ratio of an aldehyde compound to (meth)acrylic acid.

3. The method according to claim 1, wherein the alcohol is an alkyl alcohol having 1 to 6 carbon atoms.

4. The method according to claim 1, wherein the alcohol is butanol.

5. The method according to claim 1, wherein the hydrazine compound or derivative thereof is hydrazine hydrate.

6. The method according to claim 1, wherein the hydrazine compound or derivative thereof is directly added to the esterification reactor, or added to the pipeline, through which (meth)acrylic acid is transferred to the esterification reactor.

7. The method according to claim 1, wherein the preparation method of (meth)acrylate is in a continuous mode.

8. The method according to claim 1, wherein the catalyst is one or more selected from the group consisting of sulfuric acid, hydrochloric acid, phosphoric acid, nitric acid, methansulfonic acid, para toluenesulfonic acid(pTSA), alkyl sulfuric acid, natural/synthetic zeolite, cation and anion exchange resins, lithium fluoride, potassium chloride, cesium chloride, calcium chloride, ferric chloride, and aluminum phosphates, heteropoly acid, tetra alkyl titanate and polymers thereof.

9. The method according to claim 1, wherein the catalyst is para toluenesulfonic acid, cation exchange resin, or anion exchange resin.

10. The method according to claim 1, wherein the catalyst is used in an amount of 0.01 to 50% by weight, based on 100% by weight of the reactants.

11. A (meth)acrylate prepared by the method according to claim 1.
